(19) United States
(12) Patent Application Publication
(10) Pub. No.: US 2006/0088851 A1
(43) Pub. Date: Apr. 27, 2006

(54) Invasion/migration gene

(75) Inventors: Mark G. Erlander, Redwood City, CA (US); Dennis S. Sgroi, Winchester, MA (US); Xiao-Jun Ma, San Diego, CA (US)

Correspondence Address: TOWNSEND AND TOWNSEND AND CREW, LLP, TWO EMBARCADERO CENTER, 8TH FLOOR, SAN FRANCISCO, CA, 94111, US

(73) Assignees: Arcturus Bioscience, Inc., Mountain View, CA (US); General Hospital Corporation, Boston, MA (US)

(21) Appl. No.: 11/145,061

(22) Filed: Jun. 3, 2005

Related U.S. Application Data
(60) Provisional application No. 60/577,085, filed on Jun. 4, 2004.

(51) Int. Cl. C12Q 1/68; G01N 33/574

(52) U.S. Cl. 435/6; 435/7.23

(57) ABSTRACT

This invention relates to the detection of increased expression from the HoxB13 (homeobox B13) gene as indicative of an invasive or metastatic cancer phenotype. The invention provides methods of detecting the level of expression from the HoxB13 gene, optionally in combination with nodal status, as an indicator of the invasive or metastatic phenotype as well as increased cellular migration and/or mobility. The invention also provides for the measurement of expression from the HoxB13 gene to assist in the determination of patient prognosis as well as clinical diagnosis and treatment.

### RELATED APPLICATIONS

This application claims benefit of priority from U.S. Provisional Patent Application 60/577,085, which is hereby incorporated by reference in its entirety as if fully set forth.

### FIELD OF THE INVENTION

This invention relates to the detection of gene expression as indicative of a cancer phenotype. In particular, increased expression from the HoxB13 (homeobox B13) gene is indicative of an invasive cancer phenotype. The invention provides methods of detecting the level of expression from the HoxB13 gene as an indicator of the invasive phenotype as well as increased cellular migration and/or mobility. The invention also provides for the measurement of expression from the HoxB13 gene to assist in clinical diagnosis and treatment, as well as the determination of patient prognosis.

### SUMMARY OF THE INVENTION

The invention provides for the determination of the level of gene expression as an indicator of particular cancer phenotypes. The invention is based in part on the discovery that increased expression from the HoxB13 gene is indicative of an invasive cancer phenotype as well as increased cellular migration and/or mobility. The invention also provides for the determination of no increased expression from the HoxB13 gene as an indicator of the absence of an invasive phenotype. The correspondence between expression of HoxB13 sequences and the presence or absence of an invasive phenotype may also be applied to diagnose and select treatment for a subject. Additionally, the correspondence may be used to determine the likely prognosis or disease outcome for a subject. Non-limiting examples of an invasive phenotype include expansion of a primary tumor mass into surrounding tissues and invasion of cells as part of metastasis to a different tissue type. In some embodiments, the invention is applied to human subjects, such as those afflicted with, or suspected of being afflicted with, cancer. In some particular embodiments, the invention is applied to cases of breast cancer in human patients.

The origins of the invention include detection of HOXB13 expression in normal cells of the terminal duct lobular unit, the anatomic substructure of the human breast from which breast cancer arises. This suggested that the homeobox protein may play a role in breast development and physiology. The level of expression from the HoxB13 gene in such normal cells may be assayed by any appropriate technique. The level of expression in such cells may be viewed as normal used as a reference for comparison to the level of expression in non-normal or abnormal cells (e.g. cancer cells) of the same type and/or tissue as described herein. Alternatively, the expression in normal cells of a particular type or tissue may be used as a reference for comparison of heterologous non-normal or abnormal cells upon determination of suitability for such use.

The invention is also based in part on the discovery that ectopic expression of HOXB13 in cells potentiates cell invasion and migration in vitro, indicating that HOXB13 contributes to tumor invasion and metastasis in vivo. The increased potential for invasion and/or migration characteristics may be stimulated by EGF in responsive cells or by the presence of collagen.

In a first aspect, the invention provides for a method of identifying or classifying one or more cells as having an increased potential for invasion and/or migration by determining the level of expression from the HoxB13 gene as being increased or above normal. This includes the potential for metastasis to a different part and/or different tissue of a subject in which the cell(s) are found. The determination of an increase or level above normal may be made by relative comparison to the level of expression in normal cells of the same type and/or tissue. The one or more cells may be in a biological sample of cells obtained from a subject afflicted with, or suspected of being afflicted with, cancer. In some embodiments of the invention, the cancer is breast cancer. In some embodiments, expression from the HoxB13 gene in cancer cells is compared with normal cells of the same type or from the same tissue; as a non-limiting example, expression in breast cancer cells is compared with that of normal breast cells.

The determination of an increase in HoxB13 expression may be relative to other appropriate measurements of HoxB13 expression. These include comparisons to HoxB13 expression in cancer cells of the same type in other subjects, or a population of such subjects. In some embodiments, the subjects are of a population with the same cancer but which did not experience metastasis or recurrence of the cancer. This can be readily performed by retrospective analysis of samples, such as fixed samples, from such subjects or a population of such subjects. The comparison may also be to a database of HoxB13 expression levels comprising expression levels from cell samples of subjects without subsequent metastasis or cancer recurrence. Another comparison may be to a threshold level of HoxB13 expression at or above which an increased likelihood of metastasis, invasiveness, and/or migration is indicated.

The invention may thus be used to identify or classify one or more cells as pre-invasive, and/or as having an increased potential for invasion and/or migration, such as metastasis to a different site. The subject from which the cell(s) were obtained may thus be identified as having pre-invasive cancer cells with this increased potential. Alternatively, the invention may be used to identify one or more cells as invasive. In some embodiments, the one or more cells are primary cancer cells, such as primary breast cancer cells, from a subject. This aspect of the invention may be used as an early predictor or indicator of the invasive or metastatic potential of primary cancer cells in the subject. It may also be used as a predictor or indicator of the potential for cancer recurrence, such as in subjects where cancer has yet to recur. Recurrence may be the result of undetected or micrometastases that are later identified as cancer recurrence, whether local, regional or distant.

In another aspect, the assessment of HoxB13 expression may be used in combination with nodal status as a combined predictor of invasive or metastatic potential. Thus the invention-provides for the determination of 1) whether a subject with at least a primary cancer has cancer that has spread to one or more lymph node and 2) the level of HoxB13 expression in one or more cells of the primary cancer. Where the subject is “node negative” (where no cancer is detected in a lymph node) and the cancer cell(s) have a high or above normal level of HoxB13 expression, the cancer cells are identified as having an increased potential for invasion, migration, and/or metastasis. The invention, however, is not limited to such a combination assessment because a “node positive” determination in combination with increased HoxB13 expression may still indicate a potential for invasion, migration, and/or metastasis.

As explained above and herein, the identification of an increased or above normal level of expression from the HoxB13 gene in one or more cells of a sample may be used to identify the cell(s) and/or the sample as having an invasive phenotype. The sample may be a cell containing biological sample obtained from a subject afflicted with, or suspected of being afflicted with, cancer, such as breast cancer. The cell(s) may also be that which is identified as atypical or pre-cancerous.

In some embodiments, the sample is from a subject afflicted with or suspected of having cancer, such as breast cancer. In some cases, the presence of cancer is already known, and the invention is used to determine whether the cancer, such as breast cancer, has an invasive phenotype. In other embodiments, the methods of the invention may be used with cell containing samples from surgical intervention, such as that which occurs in some breast cancer patients, to determine whether the breast cancer had an invasive phenotype such that the likelihood of, or potential for, metastasis (local, regional or distant) is increased.

The methods of the invention may be advantageously applied to cells that are responsive to epidermal growth factor (EGF), insulin, glucocorticoids, and cholera endotoxin such that the invasion and/or migration characteristics of the cells are enhanced in the presence of EGF and these other factors. The determination of responsiveness to EGF may be made by any appropriate method, including the detection of expression of a receptor for EGF in said cells. Methods for the detection of EGF receptor expression, mutant EGF receptor expression, as well as EGF receptor gene amplification are known and include, but are not limited to, methods such as detection of receptor mRNA expression, detection of receptor protein expression, detection of receptor gene amplification, and detection of the expression of one or more genes which are expressed in correlation with the EGF receptor. Such methods may be used in combination with the methods disclosed herein to identify cells as being enhanced in invasiveness in the presence of EGF due to both an increased level of expression from the HoxB13 gene and positive EGF receptor expression.

The methods of the invention may comprise determination of the level of expression from the HoxB13 gene by assaying for expressed nucleic acid molecules or expressed polypeptide molecules corresponding to the HoxB13 gene. Thus assays based upon detection of transcription or translation levels, as well as stability of nucleic acid or polypeptide molecules may be used in the practice of the invention. The invention may be practiced by detecting the expression of any transcribed sequence from the HoxB13 gene. Assays for the demethylation of the HoxB13 gene, as an indicator of DNA status for HoxB13 expression, may also be used.

Thus the invention may be practiced by detection of a portion of the nucleic acid or polypeptide molecules expressed from the HoxB13 gene. Various methods for the detection of gene expression, and the expression of HoxB13 sequences expressed in transcripts from the HoxB13 gene are disclosed in U.S. applications Ser. No. 06/504,087, filed Sep. 19, 2003, Ser. No. 10/727,100, filed Dec. 2, 2003, and Ser. No. 10/773,761, filed Feb. 6, 2004 (all three of which are hereby incorporated by reference as if fully set forth) and may be used in the practice of the instant invention. Briefly, the expression of all or part of a HoxB13 transcript may be detected by use of hybridization mediated detection (such as, but not limited to, microarray, bead, or particle based technology) or quantitative PCR mediated detection (such as, but not limited to, real time PCR and reverse transcriptase PCR) as non-limiting examples. The expression of all or part of HOXB13 polypeptide may be detected by use of immunohistochemistry techniques or other antibody mediated detection (such as, but not limited to, use of labeled antibodies that bind specifically to at least part of a HOXB13 polypeptide relative to other polypeptides) as non-limiting examples. Accordingly, the invention may be practiced by detecting the expression of all or part of the HoxB13 nucleic acid and/or polypeptide sequences disclosed in the above three applications as well as sequences known in the art or as come within the knowledge in the art. It should be noted that as provided in those applications, HoxB13 expression is decreased in breast cancer cells that are responsive or sensitive to tamoxifen treatment while increased in breast cancer cells that are resistant or insensitive to tamoxifen treatment.

In a further aspect of the invention, the methods disclosed herein may be used to identify or classify one or more cells of a sample as not having an increased potential for invasion and/or migration characteristics based upon the lack of an increase in the level of expression from the HoxB13 gene or the absence of such an increase in comparison to the level of expression in normal cells of the same type and/or tissue. Such methods may also be used to identify one or more cells as not being invasive or being pre-invasive without an increased potential for invasion and/or migration.

In yet another aspect, the methods of the invention may be applied to assist in the diagnosis and treatment of a subject with cancer. The methods disclosed herein may be used to establish a diagnosis of invasive or non-invasive cancer, such as invasive or non-invasive breast cancer, in a subject from which a cell containing sample is obtained and assayed for HoxB13 expression levels. Within the category of non-invasive breast cancer, the methods may be used to establish a diagnosis of pre-invasive cancer, such as pre-invasive breast cancer, with an increased potential for invasion and/or migration. Using breast cancer as a non-limiting example, the invention may be used with cells that are identified as ADH or DCIS to identify them as pre-invasive, but with increased potential for invasion and/or migration, such as seen in the case of IDC. More generally, the methods of the invention may be used to diagnose or identify cells, including cancer or pre-cancer cells that are not known to have an increased potential for metastasis, as pre-metastatic and/or having an increased potential for metastasis. The assaying for HoxB13 expression levels may be performed as part of immunohistochemistry techniques (and/or fluorescence in situ hybridization) used in standard clinical pathology protocols to analyze a cell containing sample or specimen. Appropriate treatments, based upon the diagnosis, can thus be selected and applied based upon use of determinations of HoxB13 expression levels.

The methods of the invention may also be used to confirm or reject a diagnosis of invasive cancer, such as invasive breast cancer or breast cancer with an increased potential for metastasis, for a subject based upon assaying HoxB13 expression levels in a cell containing sample as disclosed herein. The confirmation or rejection may be of an initial diagnosis based upon use of standard clinical pathology techniques (without detection of HoxB13 expression), such as immunohistochemistry and visual inspection of cell containing samples from a subject. The present invention thus provides an improvement in the ability to obtain a more accurate diagnosis with reference to cancer invasiveness over previous protocols.

The selection of treatment based upon a diagnosis, that relies in whole or in part on the level of expression from the HoxB13 gene, includes the avoidance or elimination of certain treatments that are less likely to be of benefit. As a non-limiting example, the accurate diagnosis of a subject as having invasive cancer or cancer with metastatic potential, as opposed to non-invasive cancer, provides support for selecting a more aggressive treatment rather than risk the use of a less aggressive treatment that permits the cancer to advance in severity. Alternatively, the accurate diagnosis of non-invasive cancer may be used to support the selection of a less aggressive treatment that spares a patient the discomfort and undesirable side effects of a more aggressive treatment.

In a yet further aspect, the invention provides a method of determining the prognosis of a subject based upon the level of expression from the HoxB13 gene as described herein. In this aspect, the correspondence of HoxB13 expression to cancer invasiveness is linked to information regarding cancer invasiveness and patient prognosis or disease outcome such that HoxB13 expression is indicative of prognosis and/or disease outcome. In non-limiting examples, the prognosis and/or disease outcome includes life expectancy, likelihood of cancer recurrence over various time intervals, and likelihood of cancer invasion and/or metastasis into other tissues, or other parts, of the patient.

The invention further provides for its use as part of the clinical or medical care of a patient. Other clinical methods include those involved in the providing of medical care to a patient based on the determinations of HoxB13 expression as described herein. In some embodiments, the methods relate to providing diagnostic services based on HoxB13 expression levels, with or without inclusion of an interpretation of the significance or implications of the levels. In some embodiments, the method of providing a diagnostic service of the invention is preceded by a determination of a need for the service. In other embodiments, the method includes acts in the monitoring of the performance of the service as well as acts in the request or receipt of reimbursement for the performance of the service.

The details of one or more embodiments of the invention are set forth in the accompanying drawing and the description below. Other features and advantages of the invention will be apparent from the drawing and detailed description, and from the claims.

### BRIEF DESCRIPTION OF THE DRAWINGS

FIG. 1 shows the relative quantitative HOXB13 gene expression values in normal (N, n=45), DCIS (n=42), and IDC (n=29) cases of breast cells. Error bars denote 95% confidence intervals.

FIG. 2 shows the results of in situ hybridization of HOXB13 mRNA. DIG11UTP-labeled RNA probes with anti-sense hybridization to human breast epithelium of (i) the normal terminal duct lobular unit (200× magnification), (ii) ductal carcinoma in situ (400× magnification) and (iii) invasive ductal carcinoma (400× magnification), and sense probe hybridization to (iv) invasive ductal carcinoma (400× magnification). Inserts represent select regions of each field at 1000× magnification. L, S, and T denote lobule, stroma and tumor, respectively.

FIG. 3 shows the results from a migration assay with cells that express HOXB13 ectopically. The mean numbers of cells that migrated through the transwell filter per 20×-field are shown (±standard deviation of triplicate wells). EGF refers to the presence of epidermal growth factor. The insert represents ectopic expression of HOXB13 in MCF10A cells; reverse transcription PCR analysis of HOXB13 from expression constructs with pBABE vector alone (lane 1) or HOXB13 (lane 2). Error bars indicate one standard deviation. The asterisk * indicates P<0.05 compared to control cells.

FIG. 4 shows the results from an invasion assay with cells that express HOXB13 ectopically. The mean number of cells that invaded is shown. EGF refers to the presence of epidermal growth factor. Error bars indicate one standard deviation. The asterisk * indicates P<0.05 compared to control cells.

FIG. 5 shows the 2D morphology of MCF10A cells with MCF10A cells that express HOXB13 ectopically.

FIG. 6 shows ectopic HoxB13 expression in MCF10A cells enhances EGF-stimulated migration through extracellular matrix components from EHS (Engelbroth-Holm-Swarm) sarcoma.

FIG. 7 shows ectopic HoxB13 expression in MCF10 cells enhances EGF-stimulated invasion through extracellular matrix components from EHS (Engelbroth-Holm-Swarm) sarcoma.

FIG. 8 shows HoxB13 expression in AN10 cells enhances migration with and without a synthetic dimerizer (AP1510). AM denotes assay medium; coll refers to collagen.

### DETAILED DESCRIPTION OF MODES OF PRACTICING THE INVENTION

This invention provides methods relating increased HoxB13 expression in cells to the phenotype of invasion and/or migration characteristics, including the phenotype of metastatic potential for cancer development in other tissues or parts of a subject afflicted with a primary cancer. Non-limiting examples of such characteristics include increased cellular mobility and/or migration and the ability to invade and/or migrate through an extracellular matrix or basement membrane, such as those in vivo or in the presence of collagen. The characteristics may also be considered an increased likelihood of invasiveness and/or metastasis, or the capability of being invasive and/or metastatic. The invention thus provides a first method for identifying or classifying one or more sample cells as having an increased potential for invasion and/or migration characteristics by determining the level of expression from the HoxB13 gene in said one or more cells, wherein a relatively increased or above normal level of expression indicates an increased potential for metastatic, invasion and/or migration characteristics in said one or more cells.

The determination of a relative increase in HoxB13 expression may be by comparison to another level of HoxB13 expression, such as those in a cells of another subject or population of subjects. The cells may be those which are not cancerous as well as cancer cells, including those which did nor did not lead to cancer metastasis or invasion. Of course the comparison may be made between identical tissue types, such as breast cancer to breast tissue, or breast cancer to breast cancer. In some embodiments, the comparison is to HoxB13 expression in cancer cells of the same type in another subject, or a population of such subjects, where the same cancer did not have the same phenotype of metastatic, invasion and/or migration characteristics as described herein. Such comparisons are readily made by use of fixed samples of cancers from subjects for whom subsequent course of disease and clinical outcomes are known. Non-limiting examples of such subsequent clinical history include metastasis or cancer recurrence. The expression levels in such samples may be considered the reference levels to which the level of HoxB13 expression in a new, test, or unknown sample is compared. These reference expression levels may be in the form of a database to which the expression level in a new, test, or unknown sample is compared. The database may also include reference expression levels of samples from subjects who did subsequently experience cancer metastasis or recurrence. These levels may also be used in comparison with the level of HoxB13 expression in a new, test, or unknown sample in the practice of the invention, in which similar levels indicate the likelihood of similar outcomes. In other non-limiting embodiments, the reference expression levels, either of subjects with or without subsequent metastasis or cancer recurrence, may be used, to derive a threshold level of HoxB13 expression levels above which an increased likelihood of metastasis, invasiveness, and/or migration is present.

The invention provides a second method for identifying or classifying one or more sample cells as being invasive by determining the level of expression from the HoxB13 gene in said one or more cells, wherein an above normal level of expression indicates that said one or more cells are invasive. A third method is provided for identifying or classifying one or more sample cells as being invasive by determining the level of expression from the HoxB13 gene in said one or more cells, wherein said cells are responsive to EGF and an above normal level of expression indicates that said one or more cells are invasive in the presence of EGF. In some embodiments, the cell(s) express a proteinaceous receptor for EGF, and the methods of the invention may comprise detection of the proteinaceous receptor in the cells assayed for HoxB13 expression.

The invention provides a fourth method for identifying or classifying one or more sample cells as not having an increased potential for invasion and/or migration characteristics, including the phenotype of metastatic potential for cancer development in other tissues or parts of a subject afflicted with a primary cancer, by determining the level of expression from the HoxB13 gene in said one or more cells, wherein a normal or below normal level of expression indicates the absence of an increased potential for metastatic, invasion and/or migration characteristics in said one or more cells. In combination with the first method above, the invention thus provides a method of identifying or classifying the potential of one or more sample cells to metastasize into other tissues. Such a method may comprise determining the level of expression from the HoxB13 gene in said one or more cells, wherein an above normal level of expression indicates an increased potential for metastasis in said one or more cells and a normal or below normal level of expression indicates the absence of an increased potential, or a decreased potential, for metastasis.

A fifth method is provided for determining the prognosis of a subject by determining the level of expression from the HoxB13 gene in one or more cells of a biological sample obtained from said subject, wherein an above normal level of expression indicates an increased potential for invasive cancer in said subject and a normal or below normal level of HoxB13 expression indicates the absence of an increased potential for invasive cancer. In a related manner, the invention provides a method of predicting the prognosis or disease outcome of a subject. Such a method may comprise determining the level of expression from the HoxB13 gene in one or more cells of a biological sample obtained from said subject, wherein an above normal level of expression indicates an increased potential for cancer metastasis, increased likelihood of cancer recurrence, or decreased life expectancy in said subject and a normal or below normal level of HoxB13 expression indicates the absence of an increased potential for cancer metastasis, increased likelihood of cancer recurrence, or decreased life expectancy.

The invention also provides for the use of the prognosis or outcome in determining the treatment of a subject. Such a method may comprise determining the prognosis or outcome of a subject as described above and determining the treatment for said subject based on said prognosis or outcome. The choice of treatment may include the avoidance or elimination of certain treatments that are less likely to be of benefit. In some cases, this may mean the selection of a more aggressive treatment where a subject has cancer with metastatic potential as opposed to non-invasive cancer. In other cases, this may mean the selection of a less aggressive treatment that spares a patient the discomfort and undesirable side effects because a subject has a non-invasive cancer.

In additional embodiments of the invention, the nodal status of a subject may be determined and used in combination with the level of HoxB13 expression in the disclosed methods. Thus the invention includes methods that comprise evaluating the nodal status of the subject, wherein the absence of cancer in the lymph nodes in combination with an above normal level of HoxB13 expression is used to indicate said increased potential for metastasis and/or invasion or migration. Such methods may be advantageously applied to reduce the likelihood of a node negative determination resulting in inadequate treatment of a patient. The invention's ability to determine whether such node negative subjects may be at increased risk of invasive or metastatic cancer, based on HoxB13 expression, allows a skilled person to determine whether a node negative status includes an increased potential for metastasis.

As recognized by the skilled person, one example of an invasive phenotype is the expansion of a primary (or original) tumor mass into surrounding tissue, without a requirement for cells to detach from a tumor mass and invade a different tissue or a different part of an organism. Another example is in the spread, or metastasis, of cancer cells from one part of an organism to another part (or different tissue). This requires cancer cells to be relocated from their initial location and then give rise to one or more secondary (or metastatic) tumors as part of the metastatic process. Thus cells of invasive ductal carcinoma are not necessarily metastatic, because they merely have the ability to expand beyond the ductal environment into surrounding breast tissue without necessarily having metastatic potential. The invention provides, however, provides for the ability to identify such cells as having metastatic potential based upon HoxB13 expression. Also as known to the skilled person, cells that have metastasized may, or may not, retain the potential to metastasize further. As such, the cells of a secondary or metastatic tumor may or may not have elevated HoxB13 expression as described herein.

In some embodiments, HoxB13 expression is measured in cells of a cancer selected from Adenocarcinoma of Breast, Adenocarcinoma of Cervix, Adenocarcinoma of Esophagus, Adenocarcinoma of Gall Bladder, Adenocarcinoma of Lung, Adenocarcinoma of Pancreas, Adenocarcinoma of Small-Large Bowel, Adenocarcinoma of Stomach, Astrocytoma, Basal Cell Carcinoma of Skin, Cholangiocarcinoma of Liver, Clear Cell Adenocarcinoma of Ovary, Diffuse Large B-Cell Lymphoma, Embryonal Carcinoma of Testes, Endometrioid Carcinoma of Uterus, Ewings Sarcoma, Follicular Carcinoma of Thyroid, Gastrointestinal Stromal Tumor, Germ Cell Tumor of Ovary, Germ Cell Tumor of Testes, Glioblastoma Multiforme, Hepatocellular Carcinoma of Liver, Hodgkin's Lymphoma, Large Cell Carcinoma of Lung, Leiomyosarcoma, Liposarcoma, Lobular Carcinoma of Breast, Malignant Fibrous Histiocytoma, Medulary Carcinoma of Thyroid, Melanoma, Meningioma, Mesothelioma of Lung, Mucinous Adenocarcinoma of Ovary, Myofibrosarcoma, Neuroendocrine Tumor of Bowel, Oligodendroglioma, Osteosarcoma, Papillary Carcinoma of Thyroid, Pheochromocytoma, Renal Cell Carcinoma of Kidney, Rhabdomyosarcoma, Seminoma of Testes, Serous Adenocarcinoma of Ovary, Small Cell Carcinoma of Lung, Squamous Cell Carcinoma of Cervix, Squamous Cell Carcinoma of Esophagus, Squamous Cell Carcinoma of Larynx, Squamous Cell Carcinoma of Lung, Squamous Cell Carcinoma of Skin, Synovial Sarcoma, T-Cell Lymphoma, or Transitional Cell Carcinoma of Bladder. In other embodiments, HoxB13 expression is measured in cells, or cancer cells, of a tissue selected from Adrenal, Bladder, Bone, Brain, Breast, Cervix, Endometrium, Esophagus, Gall Bladder, Kidney, Larynx, Liver, Lung, Lymph Node, Ovary, Pancreas, Prostate, Skin, Soft Tissue, Small/Large Bowel, Stomach, Testes, Thyroid, or Uterus.

The invention is based in part on two discoveries. The first is that HoxB13 expression is increased in pre-invasive and invasive primary cancers, such as breast cancer and melanoma. The second is that ectopic expression of HOX13 in MCF10 and AN10 cells potentiates cell migration and invasion, indicating that HOXB13 expression contributes to tumor invasion and metastasis. MCF10A cells are available from the ATCC (American Type Culture Collection) under the number CRL-10317. MCF10 A is a non-transformed, non-tumorigenic, mammary epithelial cell line which responds to insulin, glucocorticoids, and EGF. The increased level of invasion and/or migration is enhanced by the presence of EGF, collagen, or AP1510, a synthetic dimerizer that increases protein-protein interactions (see Amara et al., Proc. Natl. Acad. Sci., USA 94:10618-10623, (1997) for a discussion of AP1510).

Without being bound by theory, and offered in the interest of improving understanding of the invention, it is noted that functional cooperation between HOXB13 and EGFR signaling pathways may be relevant in the context of tamoxifen resistance because activation of growth factor signaling pathways (EGFR, ERBB2) can cause tamoxifen-resistant tumor growth (see Nicholson et al. “Epidermal growth factor receptor expression in breast cancer: association with response to endocrine therapy.”Breast Cancer Res. Treat. 29:117-25 (1994) and Dowsett “Overexpression of HER-2 as a resistance mechanism to hormonal therapy for breast cancer.”Endocr. Relat. Cancer 8:191-5 (2001)). Given the known role of ERBB2 overexpression in human breast cancer, the apparent in vitro interaction between HOXB13 expression and EGF signaling pathways may point to possible therapeutic options in tumors with high expression of HOXB13 . In fact, targeting the ERBB2 pathway through blocking antibodies (Herceptin) has been suggested in the context of tamoxifen resistance based on the link between activation of growth factor signaling pathways and estrogen-independent tumor growth. HOXB13 may also have a direct effect on ER signaling, since homeobox proteins have been shown to inhibit the histone acetyltransferase activity of CBP/p300 (see Shen et al. “The HOX homeodomain proteins block CBP histone acetyltransferase activity.” Mol Cell Biol 21:7509-22 (2001)), a key co-activator for ER-dependent transcriptional regulation (see Chakravarti et al. “Role of CBP/P300 in nuclear receptor signaling.” Nature 383:99-103 (1996) and Hanstein et al. “p300 is a component of an estrogen receptor coactivator complex.”Proc Natl Acad Sci., USA 93:11540-5 (1996)). Therefore, HOXB13 may be involved directly or indirectly in the modulation of ER signaling pathways, a possibility that is of particular interest given its clinical correlation with tamoxifen resistance.

Thus the invention also provides methods to predict the treatment outcome of therapies directed at sensitivity or responsiveness of a cancer, such as breast cancer or melanoma, to estrogen or EGF. In some embodiments, the method comprises determining the level of HoxB13 expression wherein an above normal expression would predict the lack of sensitivity or responsiveness to therapies directed at the estrogen receptor (e.g. use of an “antiestrogen” agent) to prevent or reduce invasion, migration, and/or metastasis by the cells. Such therapies include treatment with one or more (or a combination of) specific estrogen receptor modulators (SERMs), like Tamoxifen; specific estrogen receptor down-regulators (SERDs); aromatase inhibitors (AIs), including nonsteroidal or steroidal agents; and irreversible inhibitors of estrogen receptor. Conversely, the lack of increased HoxB13 expression would predict the efficacy of such therapies to prevent or reduce invasion, migration, and/or metastasis by the cells.

Aromatase is an enzyme that provides a major source of estrogen in body tissues including the breast, liver, muscle and fat. Examples of nonsteroidal AIs, which inhibit aromatase via the heme prosthetic group) include, but are not limited to, anastrozole (arimidex), letrozole (femara), and vorozole (rivisor). Examples of steroidal AIs, which inactivate aromatase, include, but are not limited to, exemestane (aromasin), androstenedione, and formestane (lentaron). Other forms of therapy to reduce estrogen levels include surgical (physical removal of the ovaries) or chemical ovarian ablation (use of agents to block ovarian production of estrogen). One non-limiting example of the latter are agonists of gonadotropin releasing hormone (GnRH), such as goserelin (zoladex).

Similarly, embodiments of the invention include a method comprising determining the level of HoxB13 expression wherein an above normal expression would predict the sensitivity or responsiveness to therapies directed at the EGF signal transduction pathway to prevent or reduce invasion, migration, and/or metastasis by the cells. Such therapies include treatment with agents that directly interact with the EGF receptor family, like erbitux, and tyrosine kinase inhibitors, like Iressa and Tarceva. Other therapies include those which target or inhibit other factors (such as proteins and enzymes as non-limiting examples) in the EGF receptor pathway. Conversely, the lack of increased HoxB13 expression would predict the lack of efficacy of such therapies to prevent or reduce invasion, migration, and/or metastasis by the cells.

Treatments like those described above maybe provided pre-operatively, such as part of neoadjuvant treatment, or post-operatively, such as adjuvant treatment. In cases of pre-operative treatments, non-limiting examples of treatment outcomes include complete, intermediate, or no response, such as those based on the “clinical response” or “pathological response”. Alternatively, outcomes may be disease regression or stable disease (such as lack of metastasis or invasion), or disease progression (such as subsequent metastasis or cancer recurrence). In cases of post-operative treatments, non-limiting examples of treatment outcomes include cancer metastasis or invasion which result in local recurrence, regional recurrence, contralateral recurrence, distant recurrence, secondary primary, and death or survival due to the cancer. Other outcomes include, in relation to metastasis or invasion, relapse free survival, disease free survival, and overall survival.

In some embodiments, the present invention may thus be advantageously used in combination with surgerical intervention (e.g. surgical removal of a tumor in whole or in part) wherein the surgically removed tissue is tested for HoxB13 expression as described herein. The expression level may be used as an indicator or predictor of the likelihood of local, regional, distant, or contralateral cancer recurrence; the occurrence of one or more metastasized tumor, such as that resulting from micrometastasis; a secondary primary; or death or survival, such as relapse or recurrence free survival, disease free survival, and overall survival.

Turning now to various non-limiting modes of practicing the invention, it should be noted that while the invention may be practiced based on the identity of human homeobox B13 (HOXB13 ), which has been mapped to human chromosome 17 at 17q21.2, and animal counterparts thereof for the determination of invasive cells in non-human animals, the invention may also be practiced with any other sequence the expression of which is correlated with the expression of HoxB13 sequences.

The expression levels of HoxB13 sequences may be used alone or in combination with other sequences capable of determining various phenotypes or characteristics of cancer cells in comparison to non-cancer or normal cells. As a non-limiting example, the invention may be practiced such that the expression levels of both HoxB13 and EGF receptor are assayed. The cells used in the practice of the invention may express a detectable amount of a proteinaceous receptor for epidermal growth factor (EGF). In some embodiments, such cells are responsive to epidermal growth factor (EGF) or are stimulated to proliferate in the presence of EGF. Alternatively, the invention may be practiced such that HoxB13 expression levels are evaluated in combination with nodal status.

In some embodiments, the expression of HoxB13 sequences is used in combination with the expression from another gene (such as, but not limited to a reference gene that is expressed at the same levels in both cancer and non-cancer or normal cells) in the same cell containing sample, such as in the format of a ratio of expression levels that can readily indicate HoxB13 expression as increased or not. Alternatively, the invention provides for ratios of the expression level of a HoxB13 sequence to the expression level of a sequence that is underexpressed in an invasive cell as a indicator of invasiveness or invasive potential; increased migration or the potential for increased migration; or increased metastatic potential. Non-limiting examples include a ratio of Hoxb13 expression to IL17br expression or a ratio of Hoxb13 to Chdh expression as set forth in U.S. application Ser. No. 10/773,761, filed Feb. 6, 2004. Of course a ratio of HoxB13 expression to the expression of other genes with expression like IL17br and Chdh in cancer cells may also be used. The skilled person would readily recognize that the measurement of HoxB13 expression levels may be used as either the numerator or denominator in such ratios without complication given the relationship between HoxB13 expression and the cancer phenotypes as described herein.

The focus on the expression of HoxB13 sequences provides a way to diagnose and/or determine treatment for a cancer afflicted subject based on objective, molecular criteria. This methodology can also be used to determine patient prognosis and likely disease outcome. The methods of the invention are an advance over the use of cytomorphology and to identify risk to patients. In some embodiments, the methods may be used in combination with assessments of relative risk of breast cancer such as that discussed by Tan-Chiu et al. (J Natl Cancer Inst. 95(4):302-307, 2003). Non-limiting examples include assaying of minimally invasive sampling, such as random (periareolar) fine needle aspirates or ductal lavage samples (and optionally in combination with or as an addition to a mammogram positive for benign or malignant breast cancer), of breast cells for the expression levels of HoxB13 sequences as disclosed herein. Other applications of the invention include assaying of advanced breast cancer, including cancer suspected of being metastatic in nature.

An assay method of the invention may utilize a means related to the expression level of a HoxB13 sequence as long as the assay reflects, quantitatively or qualitatively, expression of the sequence. In some embodiments, a quantitative assay means is used. The ability to determine metastatic, invasiveness and/or migration characteristics is provided by the recognition of the relevancy of the level of expression of HoxB13 and not by the form of the assay used to determine the actual level of expression. Identifying features of the sequences include, but are not limited to, unique nucleic acid sequences used to encode (DNA), or express (RNA), a HoxB13 sequence or epitopes specific to, or activities of, proteins encoded by a HoxB13 sequence. Another means is by demethylation of HoxB13 encoding DNA, which is normally methylated in many tissues, as an indicator of increased expression.

Alternative means include detection of nucleic acid amplification as indicative of increased expression levels and nucleic acid inactivation, deletion, or methylation, as indicative of decreased expression levels. Stated differently, the invention may be practiced by assaying one or more aspects of the DNA template(s) underlying the expression of HoxB13 sequence(s), of the RNA used as an intermediate to express the sequence(s), or of the proteinaceous product expressed by the sequence(s), as well as proteolytic fragments of such products. As such, the detection of the presence of, amount of, stability of, or degradation (including rate) of, such DNA, RNA and proteinaceous molecules may be used in the practice of the invention. Of course a measurement of any HoxB13 nucleic acid molecule may be conducted by use of hybridization to a probe sequence as a non-limiting example.

Furthermore, the function, or post-translational modification, of a HoxB13 encoded product may be assayed as an indicator of expression. Non-limiting examples include measurement of HOXB13 protein interaction with one or more binding partners or of covalent modification of a HOXB13 polypeptide, such as, but not limited to, phosphorylation, glycosylation, or acylation. As would be appreciated by the skilled person, measuring the expression level of HoxB13 may be used to define a population of subjects or patients into at least two populations, such as above and below an expression level, such as that of a normal cell.

The practice of the present invention is unaffected by the presence of minor mismatches between a particular HoxB13 sequence and those expressed by cells of a subject's sample. A non-limiting example of the existence of such mismatches is seen in the case of sequence polymorphisms between individuals of a species, such as individual human patients within Homo sapiens. Knowledge that expression of a HoxB13 sequence (and sequences that vary due to minor mismatches) is correlated with the invasiveness and/or migration phenotype is sufficient for the practice of the invention with an appropriate cell containing sample via an assay for expression.

In some embodiments, a cell containing sample used in the present invention contains single cells or homogenous cell populations which have been dissected away from, or otherwise isolated or purified from, contaminating cells beyond that possible by a simple biopsy. The cells may be from any source, such as, but not limited to any fluid containing, cell containing, or tissue containing sample from an organism such as a human being. Other non-limiting examples include biopsies, such as a pre-cancerous biopsy or a cancer-diagnosed biopsy. The cell(s) may also be those identified as atypical or pre-cancerous, but which use with the present invention allows for the identification of an invasive phenotype as described herein.

Methods of isolating cells are known in the art and include microdissection, laser capture microdissection (LCM), or laser microdissection (LMD). Alternatively, undissected cells within a “section” of tissue may be used. Multiple means for such analysis are available, including detection of expression within an assay for global, or near global, gene expression in a sample (e.g. as part of a gene expression profiling analysis such as on a microarray) or by specific detection, such as quantitative PCR (Q-PCR), or real time quantitative PCR. Other non-limiting measurement techniques include those based upon mass spectroscopy.

In further embodiments, the sample is isolated via non-invasive or minimally invasive means. In some embodiments of the invention, the sample contains one or more breast cancer cells selected from atypical ductal hyperplasia (ADH), ductal carcinoma in situ (DCIS), and invasive ductal carcinoma (IDC). The expression of a HoxB13 sequence(s) in the sample may be determined and compared to the expression of said sequence(s) in reference data of normal or non-cancer cells. In some cases, the reference data is obtained from the same sample or subject. In embodiments of the invention utilizing Q-PCR, the expression level may be compared to expression levels of one or more reference genes in the same sample or a ratio of expression levels (such as one based on ΔCt as a non-limiting example) may be used. Thus the invention may readily be used to identify ADH, DCIS, and/or IDC cells as having a cancer phenotype, such as metastatic potential, as described herein. This also advantageously allows for the identification of ADH or DCIS cells as having invasive potential or propensity.

When individual breast or cancer cells are isolated in the practice of the invention, one benefit is that contaminating, non-breast or non-cancer cells (such as infiltrating lymphocytes or other immune system cells) are not present to possibly affect detection of expression of the HoxB13 sequence(s).

While the present invention is described mainly in the context of human breast cancer, it may be practiced in the context of any human cancer or the cancer of any animal. Preferred animals for the application of the present invention are mammals, particularly those important to agricultural applications (such as, but not limited to, cattle, sheep, horses, and other “farm animals”), and animals for human companionship (such as, but not limited to, dogs and cats).

As used herein, a “gene” is a polynucleotide that encodes a discrete product, whether RNA or proteinaceous in nature. It is appreciated that more than one polynucleotide may be capable of encoding a discrete product. The term includes alleles and polymorphisms of a gene that encodes the same product, or a functionally associated (including gain, loss, or modulation of function) analog thereof, based upon chromosomal location and ability to recombine during normal mitosis.

A “sequence” or “gene sequence” as used herein is a nucleic acid molecule or polynucleotide composed of a discrete order of nucleotide bases. The term includes the ordering of bases that encodes a discrete product (i.e. “coding region”), whether RNA or proteinaceous in nature. It is appreciated that more than one polynucleotide may be capable of encoding a discrete product. It is also appreciated that alleles and polymorphisms of the disclosed HoxB13 sequences may exist and may be used in the practice of the invention to identify the expression level(s) of the disclosed HoxB13 sequences or an allele or polymorphism thereof. Identification of an allele or polymorphism depends in part upon chromosomal location and ability to recombine during mitosis.

The terms “correlate” or “correlation” or equivalents thereof refer to an association between expression of one or more genes and a physiological phenotype or characteristic.

A “polynucleotide” is a polymeric form of nucleotides of any length, either ribonucleotides or deoxyribonucleotides. This term refers only to the primary structure of the molecule. Thus, this term includes double- and single-stranded DNA and RNA. It also includes known types of modifications including labels known in the art, methylation, “caps”, substitution of one or more of the naturally occurring nucleotides with an analog, and internucleotide modifications such as uncharged linkages (e.g., phosphorothioates, phosphorodithioates, etc.), as well as unmodified forms of the polynucleotide.

The term “amplify” is used in the broad sense to mean creating an amplification product can be made enzymatically with DNA or RNA polymerases. “Amplification,” as used herein, generally refers to the process of producing multiple copies of a desired sequence, particularly those of a sample. “Multiple copies” mean at least 2 copies. A “copy” does not necessarily mean perfect sequence complementarity or identity to the template sequence. Methods for amplifying mRNA are generally known in the art, and include reverse transcription PCR (RT-PCR) and those described in U.S. patent application Ser. No. 10/062,857 (filed on Oct. 25, 2001), as well as U.S. Provisional Patent Applications 60/298,847 (filed Jun. 15, 2001) and 60/257,801 (filed Dec. 22, 2000), all of which are hereby incorporated by reference in their entireties as if fully set forth. Another method which may be used is quantitative PCR (or Q-PCR). Alternatively, RNA may be directly labeled as the corresponding cDNA by methods known in the art.

By “corresponding”, it is meant that a nucleic acid molecule shares a substantial amount of sequence identity with another nucleic acid molecule. Substantial amount means at least 95%, usually at least 98% and more usually at least 99%, and sequence identity is determined using the BLAST algorithm, as described in Altschul et al. (1990), J. Mol. Biol. 215:403-410 (using the published default setting, i.e. parameters w=4, t=17).

A “microarray” is a linear or two-dimensional or three dimensional (and solid phase) array of preferably discrete regions, each having a defined area, formed on the surface of a solid support such as, but not limited to, glass, plastic, or synthetic membrane. The density of the discrete regions on a microarray is determined by the total numbers of immobilized polynucleotides to be detected on the surface of a single solid phase support, preferably at least about 50/cm², more preferably at least about 100/cm², even more preferably at least about 500/cm₂, but preferably below about 1,000/cm². Preferably, the arrays contain less than about 500, about 1000, about 1500, about 2000, about 2500, or about 3000 immobilized polynucleotides in total. As used herein, a DNA microarray is an array of oligonucleotide or polynucleotide probes placed on a chip or other surfaces used to hybridize to amplified or cloned polynucleotides from a sample. Since the position of each particular group of probes in the array is known, the identities of a sample polynucleotides can be determined based on their binding to a particular position in the microarray. As an alternative to the use of a microarray, an array of any size may be used in the practice of the invention, including an arrangement of one or more position of a two-dimensional or three dimensional arrangement in a solid phase to detect expression of a single gene sequence. In some embodiments, a microarray for use with the present invention may be prepared by photolithographic techniques (such as synthesis of nucleic acid probes on the surface from the 3′ end) or by nucleic synthesis followed by deposition on a solid surface.

Because the invention relies upon the identification of gene expression, one embodiment of the invention involves determining expression by hybridization of mRNA, or an amplified or cloned version thereof, of a sample cell to a polynucleotide that is unique to a particular HoxB13 sequence. Preferred polynucleotides of this type contain at least about 16, at least about 18, at least about 20, at least about 22, at least about 24, at least about 26, at least about 28, at least about 30, or at least about 32 consecutive basepairs of a HoxB13 sequence that is not found in other gene sequences. The term “about” as used in the previous sentence refers to an increase or decrease of 1 from the stated numerical value. Even more preferred are polynucleotides of at least or about 50, at least or about 100, at least about or 150, at least or about 200, at least or about 250, at least or about 300, at least or about 350, at least or about 400, at least or about 450, or at least or about 500 consecutive bases of a sequence that is not found in other gene sequences. The term “about” as used in the preceding sentence refers to an increase or decrease of 10% from the stated numerical value. Longer polynucleotides may of course contain minor mismatches (e.g. via the presence of mutations) which do not affect hybridization to the nucleic acids of a sample. Such polynucleotides may also be referred to as polynucleotide probes that are capable of hybridizing to sequences of the genes, or unique portions thereof, described herein. Such polynucleotides may be labeled to assist in their detection. Preferably, the sequences are those of mRNA encoded by the genes, the corresponding cDNA to such mRNAs, and/or amplified versions of such sequences. In preferred embodiments of the invention, the polynucleotide probes are immobilized on an array, other solid support devices, or in individual spots that localize the probes.

In another embodiment of the invention, all or part of a HoxB13 sequence may be amplified and detected by methods such as the polymerase chain reaction (PCR) and variations thereof, such as, but not limited to, quantitative PCR (Q-PCR), reverse transcription PCR (RT-PCR), and real-time PCR (including as a means of measuring the initial amounts of mRNA copies for each sequence in a sample), optionally real-time RT-PCR or real-time Q-PCR. Such methods would utilize one or two primers that are complementary to portions of a HoxB13 sequence, where the primers are used to prime nucleic acid synthesis. The newly synthesized nucleic acids are optionally labeled and may be detected directly or by hybridization to a polynucleotide of the invention. The newly synthesized nucleic acids may be contacted with polynucleotides (containing sequences) of the invention under conditions which allow for their hybridization. Additional methods to detect the expression of expressed nucleic acids include RNAse protection assays, including liquid phase hybridizations, and in situ hybridization of cells.

Alternatively, and in yet another embodiment of the invention, HoxB13 expression may be determined by analysis of expressed protein in a cell sample of interest by use of one or more antibodies specific for one or more epitopes of individual HoxB13 gene products (proteins), or proteolytic fragments thereof, in said cell sample or in a bodily fluid of a subject. The cell sample may be one of breast cancer epithelial cells enriched from the blood of a subject, such as by use of labeled antibodies against cell surface markers followed by fluorescence activated cell sorting (FACS). Such antibodies are preferably labeled to permit their easy detection after binding to the gene product. Detection methodologies suitable for use in the practice of the invention include, but are not limited to, immunohistochemistry of cell containing samples or tissue, enzyme linked immunosorbent assays (ELISAs) including antibody sandwich assays of cell containing tissues or blood samples, mass spectroscopy, and immuno-PCR.

The terms “label” or “labeled” refer to a composition capable of producing a detectable signal indicative of the presence of the labeled molecule. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means.

The term “support” refers to conventional supports such as beads, particles, dipsticks, fibers, filters, membranes and silane or silicate supports such as glass slides.

The concept of a cell containing sample from the breast refers to a sample of breast tissue or fluid isolated from an individual afflicted with, suspected of being afflicted with, or at risk of developing, breast cancer. Such samples are primary isolates (in contrast to cultured cells) and may be collected by any non-invasive or minimally invasive means, including, but not limited to, ductal lavage, fine needle aspiration, needle biopsy, the devices and methods described in U.S. Pat. No. 6,328,709, or any other suitable means recognized in the art. Alternatively, the “sample” may be collected by an invasive method, including, but not limited to, surgical biopsy.

Non-limiting examples of cell containing samples for use in the invention includes fluid samples, such as blood, serum, or plasma; samples enriched for epithelial cells, endothelial cells, circulating tumor cells, or any cell of interest; fluids that contain cells and/or proteins, DNA, or RNA, such as urine or bladder washes, or a cell pellet or spread thereof, cervical scraps (e.g. PAP smears); endometrial scraps; stool; buccal cells; cell containing aspirates, such as those from any bodily mass, including a tumor mass; cell containing exfoliates; and tissue samples, such as fine needle aspirates of tissues, needle biopsies, excisional biopsies, and ThinPrep from Cytyc. In some embodiments, the sample is of a primary (original) cancer or tumor in a subject or patient. The tumor may be optionally estrogen receptor positive.

“Expression” and “gene expression” include transcription and/or translation of nucleic acid material.

As used herein, the term “comprising” and its cognates are used in their inclusive sense; that is, equivalent to the term “including” and its corresponding cognates.

Conditions that “allow” an event to occur or conditions that are “suitable” for an event to occur, such as hybridization, strand extension, and the like, or “suitable” conditions are conditions that do not prevent such events from occurring. Thus, these conditions permit, enhance, facilitate, and/or are conducive to the event. Such conditions, known in the art and described herein, depend upon, for example, the nature of the nucleotide sequence, temperature, and buffer conditions. These conditions also depend on what event is desired, such as hybridization, cleavage, strand extension or transcription.

Sequence “mutation,” as used herein, refers to any sequence alteration in the sequence of a gene disclosed herein interest in comparison to a reference sequence. A sequence mutation includes single nucleotide changes, or alterations of more than one nucleotide in a sequence, due to mechanisms such as substitution, deletion or insertion. Single nucleotide polymorphism (SNP) is also a sequence mutation as used herein. Because the present invention is based on the relative level of gene expression, mutations in non-coding regions of genes as disclosed herein may also be assayed in the practice of the invention.

“Detection” or “detecting” includes any means of detecting, including direct and indirect detection of gene expression and changes therein. For example, a “detectable increase” of a product may be observed directly or indirectly, and the term indicates any increase. A “detectable decrease” of a product may be observed directly or indirectly, and the term indicates any decrease (including the absence of detectable signal).

Increases and decreases in expression of a HoxB13 sequence are defined in the following terms based upon percent or fold changes over expression in normal cells. Increases may be of 10, 20, 30, 40, 50, 60, 70, 80, 90, 100, 120, 140, 160, 180, or 200% relative to expression levels in normal cells. Alternatively, fold increases may be of 1, 1.5, 2, 2.5, 3, 3.5, 4, 4.5, 5, 5.5, 6, 6.5, 7, 7.5, 8, 8.5, 9, 9.5, or 10 fold over expression levels in normal cells. Decreases may be of 10, 20, 30, 40, 50, 55, 60, 65, 70, 75, 80, 82, 84, 86, 88, 90, 92, 94, 96, 98, 99 or 100% relative to expression levels in normal cells.

A “selective estrogen receptor modulator” or SERM is an “antiestrogen” agent that in some tissues act like estrogens (agonist) but block estrogen action in other tissues (antagonist). A “selective estrogen receptor downregulators” (or “SERD”s) or “pure” antiestrogens includes agents which block estrogen activity in all tissues. See Howell et al. (Best Bractice & Res. Clin. Endocrinol. Metab. 18(1):47-66, 2004). Preferred SERMs of the invention are those that are antagonists of estrogen in breast tissues and cells, including those of breast cancer. Non-limiting examples of such include TAM, raloxifene, GW5638, and ICI 182,780. The possible mechanisms of action by various SERMs have been reviewed (see for example Jordan et al., 2003, Breast Cancer Res. 5:281-283; Hall et al., 2001, J. Biol. Chem. 276(40):36869-36872; Dutertre et al. 2000, J. Pharmacol. Exp. Therap. 295(2):431-437; and Wijayaratne et al., 1999, Endocrinology 140(12):5828-5840). Other non-limiting examples of SERMs in the context of the invention include triphenylethylenes, such as tamoxifen, GW5638, TAT-59, clomiphene, toremifene, droloxifene, and idoxifene; benzothiophenes, such as arzoxiphene (LY353381 or LY353381-HCl); benzopyrans, such as EM-800; naphthalenes, such as CP-336,156; and ERA-923.

Non-limiting examples of SERD or “pure” antiestrogens include agents such as ICI 182,780 (fulvestrant or faslodex) or the oral analogue SR16243 and ZK 191703 as well as aromatase inhibitors and chemical ovarian ablation agents as described herein.

Other agents encompassed by SERM as used herein include progesterone receptor inhibitors and related drugs, such as progestomimetics like medroxyprogesterone acetate, megace, and RU-486; and peptide based inhibitors of ER action, such as LH-RH analogs (leuprolide, zoladex, [D-Trp6]LH-RH), somatostatin analogs, and LXXLL motif mimics of ER as well as tibolone and resveratrol. As noted above, preferred SERMs of the invention are those that are antagonist of estrogen in sensitive tissues and cells, including those of breast cancer. Non-limiting examples of preferred SERMs include the actual or contemplated metabolites (in vivo) of any SERM, such as, but not limited to, 4-hydroxytamoxifen (metabolite of tamoxifen), EM652 (or SCH 57068 where EM-800 is a prodrug of EM-652), and GW7604 (metabolite of GW5638). See Willson et al. (1997, Endocrinology 138(9):3901-3911) and Dauvois et al. (1992, Proc. Nat'l. Acad. Sci., USA 89:4037-4041) for discussions of some specific SERMs.

Other preferred SERMs are those that produce the same relevant gene expression profile as tamoxifen or 4-hydroxytamoxifen. One example of means to identify such SERMs is provided by Levenson et al. (2002, Cancer Res. 62:4419-4426).

Unless defined otherwise all technical and scientific terms used herein have the same meaning as commonly understood to one of ordinary skill in the art to which this invention belongs.

To determine the (increased or decreased) expression levels of HoxB13 in the practice of the present invention, any method known in the art may be utilized. In one preferred embodiment of the invention, expression based on detection of RNA which hybridizes to the genes identified and disclosed herein is used. This is readily performed by any RNA detection or amplification+detection method as described herein or known or recognized as equivalent in the art such as, but not limited to, methods to detect the presence, or absence, of RNA stabilizing or destabilizing sequences.

Alternatively, expression based on detection of DNA status may be used. Detection of the HoxB13 gene as methylated or deleted may be used to detect decreased expression. The status of the promoter regions of HOXB13 may be assayed as an indication of decreased expression of HOXB13 sequences. A non-limiting example is the methylation status of sequences found in the promoter region. Conversely, detection of the HoxB13 gene as amplified may be used for genes that have increased expression in correlation with a particular breast cancer outcome. These methods may be readily performed by PCR based, fluorescent in situ hybridization (FISH) and chromosome in situ hybridization (CISH) methods known in the art.

Expression based on detection of a presence, increase, or decrease in HOXB13 protein levels or activity may also be used. Detection may be performed by any immunohistochemistry (IHC) based, bodily fluid based (where a HOXB13 polypeptide or fragment thereof is found in a bodily fluid, such as but not limited to blood), antibody (including autoantibodies against the protein where present) based, exfoliate cell (from the cancer) based, mass spectroscopy based, and image (including used of labeled ligand) based method known in the art and recognized as appropriate for the detection of the protein. Antibody and image based, methods are additionally useful for the localization of tumors after determination of cancer by use of cells obtained by a non-invasive procedure (such as ductal lavage or fine needle aspiration), where the source of the cancerous cells is not known. A labeled antibody or ligand may be used to localize the carcinoma(s) within a patient or to assist in the enrichment of exfoliated cancer cells from a bodily fluid.

Antibodies for use in such methods of detection include polyclonal antibodies, optionally isolated from naturally occurring sources where available, and monoclonal antibodies, including those prepared by use of HOXB13 polypeptides or fragment thereof as antigens. Such antibodies, as well as fragments thereof (including but not limited to Fab fragments) function to detect or diagnose non-normal or cancer cells by virtue of their ability to specifically bind HOXB13 polypeptides to the exclusion of other polypeptides to produce a detectable signal. Recombinant, synthetic, and hybrid antibodies with the same ability may also be used in the practice of the invention. Antibodies may be readily generated by immunization with a HOXB13 polypeptide or fragment thereof, and polyclonal sera may also be used in the practice of the invention.

Antibody based detection methods are well known in the art and include sandwich and ELISA assays as well as Western blot and flow cytometry based assays as non-limiting examples. Samples for analysis in such methods include any that contain HOXB13 polypeptides. Non-limiting examples include those containing breast cells and cell contents as well as bodily fluids (including blood, serum, saliva, lymphatic fluid, as well as mucosal and other cellular secretions as non-limiting examples) that contain the polypeptides.

A preferred embodiment using a nucleic acid based assay to determine expression is by immobilization of one or more HoxB13 sequences on a solid support, including, but not limited to, a solid substrate as an array or to beads or bead based technology as known in the art. Alternatively, solution based expression assays known in the art may also be used. The immobilized HoxB13 gene(s) may be in the form of polynucleotides that are unique or otherwise specific to HoxB13 such that the polynucleotide would be capable of hybridizing to a HoxB13 DNA or RNA. These polynucleotides may be the full length of the HoxB13 gene(s) or be short sequences of the genes (up to one nucleotide shorter than the full length sequence known in the art by deletion from the 5′ or 3′ end of the sequence) that are optionally minimally interrupted (such as by mismatches or inserted non-complementary basepairs) such that hybridization with a DNA or RNA corresponding to HoxB13 is not affected. Preferably, the polynucleotides used are from the 3′ end of the gene, such as within about 350, about 300, about 250, about 200, about 150, about 100, or about 50 nucleotides from the polyadenylation signal or polyadenylation site of a gene or expressed sequence. Polynucleotides containing mutations relative to the sequences of the disclosed genes may also be used so long as the presence of the mutations still allows hybridization to produce a detectable signal.

The immobilized HoxB13 gene(s) may be used to determine the state of nucleic acid samples prepared from sample breast cell(s) for which the outcome of the sample's subject (e.g. patient from whom the sample is obtained) is not known or for confirmation of an outcome that is already assigned to the sample's subject. Without limiting the invention, such a cell may be from a patient with ER+ or ER− breast cancer. The immobilized polynucleotide(s) need only be sufficient to specifically hybridize to the corresponding nucleic acid molecules derived from the sample under suitable conditions.

As will be appreciated by those skilled in the art, some HoxB13 sequences include 3′ poly A (or poly T on the complementary strand) stretches that do not contribute to the uniqueness of the disclosed sequences. The invention may thus be practiced with HoxB13 sequences lacking the 3′ poly A (or poly T) stretches. The uniqueness of the disclosed sequences refers to the portions or entireties of the sequences which are found only in nucleic acids, including unique sequences found at the 3′ untranslated portion thereof. Preferred unique sequences for the practice of the invention are those which contribute to the consensus sequences for HoxB13 such that the unique sequences will be useful in detecting expression in a variety of individuals rather than being specific for a polymorphism present in some individuals. Alternatively, sequences unique to an individual or a subpopulation may be used. The preferred unique sequences are preferably of the lengths of polynucleotides of the invention as discussed herein.

In particularly preferred embodiments of the invention, polynucleotides having sequences present in the 3′ untranslated and/or non-coding regions of HoxB13 sequences are used to detect expression levels in cancer cells or breast cancer cells in the practice of the invention. Such polynucleotides may optionally contain sequences found in the 3′ portions of the coding regions of HoxB13 sequences. Polynucleotides containing a combination of sequences from the coding and 3′ non-coding regions preferably have the sequences arranged contiguously, with no intervening heterologous sequence(s).

Alternatively, the invention may be practiced with polynucleotides having sequences present in the 5′ untranslated and/or non-coding regions of HOXB13 sequences to detect the level of expression in cancer cells or breast cancer cells. Such polynucleotides may optionally contain sequences found in the 5′ portions of the coding regions. Polynucleotides containing a combination of sequences from the coding and 5′ non-coding regions preferably have the sequences arranged contiguously, with no intervening heterologous sequence(s). The invention may also be practiced with sequences present in the coding regions of HOXB13 sequences.

Preferred polynucleotides contain sequences from 3′ or 5′ untranslated and/or non-coding regions of at least about 16, at least about 18, at least about 20, at least about 22, at least about 24, at least about 26, at least about 28, at least about 30, at least about 32, at least about 34, at least about 36, at least about 38, at least about 40, at least about 42, at least about 44, or at least about 46 consecutive nucleotides. The term “about” as used in the previous sentence refers to an increase or decrease of 1 from the stated numerical value. Even more preferred are polynucleotides containing sequences of at least or about 50, at least or about 100, at least about or 150, at least or about 200, at least or about 250, at least or about 300, at least or about 350, or at least or about 400 consecutive nucleotides. The term “about” as used in the preceding sentence refers to an increase or decrease of 10% from the stated numerical value.

Sequences from the 3′ or 5′ end of HoxB13 coding regions as found in polynucleotides of the invention are of the same lengths as those described above, except that they would naturally be limited by the length of the coding region. The 3′ end of a coding region may include sequences up to the 3′ half of the coding region. Conversely, the 5′ end of a coding region may include sequences up the 5′ half of the coding region. Of course the above described sequences, or the coding regions and polynucleotides containing portions thereof, may be used in their entireties.

In another embodiment of the invention, polynucleotides containing deletions of nucleotides from the 5′ and/or 3′ end of HoxB13 sequences may be used. The deletions are preferably of 1-5, 5-10, 10-15, 15-20, 20-25, 25-30, 30-35, 35-40, 40-45, 45-50, 50-60, 60-70, 70-80, 80-90, 90-100, 100-125, 125-150, 150-175, or 175-200 nucleotides from the 5′ and/or 3′ end, although the extent of the deletions would naturally be limited by the length of the sequences and the need to be able to use the polynucleotides for the detection of expression levels.

Other polynucleotides of the invention from the 3′ end of HoxB13 sequences include those of primers and optional probes for quantitative PCR. Preferably, the primers and probes are those which amplify a region less than about 350, less than about 300, less than about 250, less than about 200, less than about 150, less than about 100, or less than about 50 nucleotides from the from the polyadenylation signal or polyadenylation site of a gene or expressed sequence.

Other polynucleotides for use in the practice of the invention include those that have sufficient homology to HoxB13 sequences to detect their expression by use of hybridization techniques. Such polynucleotides preferably have about or 95%, about or 96%, about or 97%, about or 98%, or about or 99% identity with HOXB13 sequences as, described herein. Identity is determined using the BLAST algorithm, as described above. The other polynucleotides for use in the practice of the invention may also be described on the basis of the ability to hybridize to polynucleotides of the invention under stringent conditions of about 30% v/v to about 50% formamide and from about 0.01M to about 0.15M salt for hybridization and from about 0.01M to about 0.15M salt for wash conditions at about 55 to about 65° C. or higher, or conditions equivalent thereto.

In a further embodiment of the invention, a population of single stranded nucleic acid molecules comprising one or both strands of a human HoxB13 sequence is provided as a probe such that at least a portion of said population may be hybridized to one or both strands of a nucleic acid molecule quantitatively amplified from RNA of a cancer, such as breast cancer, cell. The population may be only the antisense strand of a human HoxB13 sequence such that a sense strand of a molecule from, or amplified from, a cancer or breast cancer cell may be hybridized to a portion of said population. The population preferably comprises a sufficiently excess amount of said one or both strands of a human HoxB13 sequence in comparison to the amount of expressed (or amplified) nucleic acid molecules containing a complementary HoxB13 sequence from a normal cell. This condition of excess permits the increased amount of nucleic acid expression in a cancer or breast cancer cell to be readily detectable as an increase.

Alternatively, the population of single stranded molecules is equal to or in excess of all of one or both strands of the nucleic acid molecules amplified from a cancer or breast cancer cell such that the population is sufficient to hybridize to all of one or both strands. Preferred cells are those of a breast cancer patient that is ER+ or for whom treatment with tamoxifen or one or more other “antiestrogen” agent against breast cancer is contemplated. The single stranded molecules may of course be the denatured form of any HoxB13 sequence containing double stranded nucleic acid molecule or polynucleotide as described herein.

The population may also be described as being hybridized to HoxB13 sequence containing nucleic acid molecules at a level of at least twice as much as that by nucleic acid molecules of a normal cell. As in the embodiments described above, the nucleic acid molecules may be those quantitatively amplified from a cancer or breast cancer cell such that they reflect the amount of expression in said cell.

The population is preferably immobilized on a solid support, optionally in the form of a location on a microarray. A portion of the population is preferably hybridized to nucleic acid molecules quantitatively amplified from a non-normal or abnormal (breast) cell by RNA amplification. The amplified RNA may be that derived from a cancer or breast cancer cell, as long as the amplification used was quantitative with respect to HoxB13 containing sequences.

In other embodiments, the nucleic acid derived from a sample cancer cell(s) may be preferentially amplified by use of appropriate primers such that only HoxB13 sequences are amplified to reduce contaminating background signals from other genes expressed in the cell. Alternatively, and where expression of other genes is also analyzed or where very few cells (or one cell) is used, the nucleic acid from the sample may be globally amplified before hybridization to the immobilized polynucleotides. Of course RNA, or the cDNA counterpart thereof, may be directly labeled and used, without amplification, by methods known in the art.

The assay embodiments described herein may be used in a number of different ways to identify or detect invasive cancers. In some cases, this would reflect a secondary screen for the patient, who may have already undergone mammography or physical exam as a primary screen. If positive from the primary screen, the subsequent needle biopsy, ductal lavage, fine needle aspiration, or other analogous minimally invasive method may provide the sample for use in the assay embodiments. The present invention is particularly useful in combination with non-invasive protocols, such as ductal lavage or fine needle aspiration, to prepare a breast cell sample.

The present invention provides a more objective set of criteria, in the form of HoxB13 expression level, to discriminate (or delineate) between (breast) cancer outcomes. In particularly preferred embodiments of the invention, the assays are used to discriminate between good and poor outcomes based upon whether a cancer is invasive. Comparisons that discriminate between outcomes after about 10, about 20, about 30, about 40, about 50, about 60, about 70, about 80, about 90, about 100, or about 150 months may be performed.

While good and poor survival outcomes may be defined relatively in comparison to each other, a “good” outcome may be viewed as a better than 50% survival rate after about 60 months post surgical intervention to remove breast cancer tumor(s). A “good” outcome may also be a better than about 60%, about 70%, about 80% or about 90% survival rate after about 60 months post surgical intervention. A “poor” outcome may be viewed as a 50% or less survival rate after about 60 months post surgical intervention to remove breast cancer tumor(s). A “poor” outcome may also be about a 70% or less survival rate after about 40 months, or about a 80% or less survival rate after about 20 months, post surgical intervention.

In one embodiment, the isolation and analysis of a breast cancer cell sample may be performed as follows:

(1) Ductal lavage or other non-invasive or minimally invasive procedure is performed on a patient to obtain a sample.

(2) Sample is prepared and coated onto a microscope slide. Note that ductal lavage results in clusters of cells that may be cytologically examined.

(3) Pathologist or image analysis software scans the sample for the presence of atypical cells.

(4) If atypical cells are observed, those cells are harvested (e.g. by microdissection such as LCM).

(5) RNA is extracted from the harvested cells.

(6) RNA is assayed, directly or after conversion to cDNA or amplification therefrom, for the expression of HoxB13 sequences.

With use of the present invention, skilled physicians may prescribe or withhold treatment with tamoxifen or another therapeutic agent against breast cancer based on prognosis determined via practice of the instant invention.

The above discussion is also applicable where a palpable lesion is detected followed by fine needle aspiration or needle biopsy of cells from the breast. The cells are plated and reviewed by a pathologist or automated imaging system which selects cells for analysis as described above.

The present invention may also be used, however, with solid tissue biopsies, including those stored as a frozen or FFPE (formalin fixed, paraffin embedded) specimen. Alternatively, a fresh or fixed sample may be obtained and used. As a non-limiting example, a solid biopsy may be collected and prepared for visualization followed by determination of expression of one or more genes identified herein to determine the (breast) cancer outcome. As another non-limiting example, a solid biopsy may be collected and prepared for visualization followed by determination of HoxB13 expression. One preferred means is by use of in situ hybridization with polynucleotide or protein identifying probe(s) for assaying HoxB13 expression. Non-limiting examples of fixed samples include those that are fixed with formalin or formaldehyde (including FFPE samples), with Boudin's, glutaldehyde, acetone, alcohols, or any other fixative, such as those used to fix cell or tissue samples for immunohistochemistry (IHC). Other examples include fixatives that precipitate cell associated nucleic acids and/or proteins. In some applications of the invention, the sample has not been classified using standard pathology techniques, such as, but not limited to, immunohistochemistry based assays.

In an alternative method, the solid tissue biopsy may be used to extract molecules followed by analysis for HoxB13. This provides the possibility of leaving out the need for visualization and collection of only cancer cells or cells suspected of being cancerous. This method may of course be modified such that only cells that have been positively selected are collected and used to extract molecules for analysis. This would require visualization and selection as a prerequisite to gene expression analysis. In the case of an FFPE sample, cells may be obtained followed by RNA extraction, amplification and detection as described herein.

The methods provided by the present invention may also be automated in whole or in part.

A further aspect of the invention provides for the use of the present invention in relation to clinical activities. In some embodiments, the determination or measurement of HoxB13 expression as described herein is performed as part of providing medical care to a patient, including the providing of diagnostic services in support of providing medical care. Thus the invention includes a method in the medical care of a patient, the method comprising determining or measuring HoxB13 expression levels in a cell containing sample obtained from a patient as described herein. The method may further comprise the interpretation of, or significance of, the determination/measurement, as indicating or predicting the presence of a cancer phenotype in a manner as described herein.

The determination or measurement of expression levels may be preceded by a variety of related actions. In some embodiments, the measurement is preceded by a determination or diagnosis of a human subject as in need of said measurement. The measurement may be preceded by a determination of a need for the measurement, such as that by a medical doctor, nurse or other health care provider or professional, or those working under their instruction, or personnel of a health insurance or maintenance organization in approving the performance of the measurement as a basis to request reimbursement or payment for the performance.

The measurement may also be preceded by preparatory acts necessary to the actual measuring. Non-limiting examples include the actual obtaining of a cell containing sample from a human subject; or receipt of a cell containing sample; or sectioning a cell containing sample; or isolating cells from a cell containing sample; or obtaining RNA from cells of a cell containing sample; or reverse transcribing RNA from cells of a cell containing sample. The sample may be any as described herein for the practice of the invention.

In additional embodiments, the invention provides for a method of ordering, or receiving an order for, the performance of a method in the medical care of a patient or other method of the invention. The ordering may be made by a medical doctor, a nurse, or other health care provider, or those working under their instruction, while the receiving, directly or indirectly, may be made by any person who performs the method(s). The ordering may be by any means of communication, including communication that is written, oral, electronic, digital, analog, telephonic, in person, by facsimile, by mail, or otherwise passes through a jurisdiction within the United States.

The invention further provides methods in the processing of reimbursement or payment for a test, such as the above method in the medical care of a patient or other method of the invention. A method in the processing of reimbursement or payment may comprise indicating that 1) payment has been received, or 2) payment will be made by another payer, or 3) payment remains unpaid on paper or in a database after performance of an expression level detection, determination or measurement method of the invention. The database may be in any form, with electronic forms such as a computer implemented database included within the scope of the invention. The indicating may be in the form of a code (such as a CPT code) on paper or in the database. The “another payer” may be any person or entity beyond that to whom a previous request for reimbursement or payment was made.

Alternative, the method may comprise receiving reimbursement or payment for the technical or actual performance of a disclosed method in the medical care of a patient; for the interpretation of the results from said method; or for any other method of the invention. Of course the invention also includes embodiments comprising instructing another person or party to receive the reimbursement or payment. The ordering may be by any communication means, including those described above. The receipt may be from any entity, including an insurance company, health maintenance organization, governmental health agency, or a patient as non-limiting examples. The payment may be in whole or in part. In the case of a patient, the payment may be in the form of a partial payment known as a co-pay.

In yet another embodiment, the method may comprise forwarding or having forwarded a reimbursement or payment request to an insurance company, health maintenance organization, governmental health agency, or to a patient for the performance of the above method in the medical care of a patient or other method of the invention. The request may be by any communication means, including those described above.

In a further embodiment, the method may comprise receiving indication of approval for payment, or denial of payment, for performance of the above method in the medical care of a patient or other method of the invention. Such an indication may come from any person or party to whom a request for reimbursement or payment was made. Non-limiting examples include an insurance company, health maintenance organization, or a governmental health agency, like Medicare or Medicaid as non-limiting examples. The indication may be by any communication means, including those described above.

An additional embodiment is where the method comprises sending a request for reimbursement for performance of the above method in the medical care of a patient or other method of the invention. Such a request may be made by any communication means, including those described above. The request may have been made to an insurance company, health maintenance organization, federal health agency, or the patient for whom the method was performed.

A further method comprises indicating the need for reimbursement or payment on a form or into a database for performance of the above method in the medical care of a patient or other method of the invention. Alternatively, the method may simply indicate the performance of the method. The database may be in any form, with electronic forms such as a computer implemented database included within the scope of the invention. The indicating may be in the form of a code on paper or in the database.

In the above methods in the medical care of a patient or other method of the invention, the method may comprise reporting the results of the method, optionally to a health care facility, a health care provider or professional, a doctor, a nurse, or personnel working therefor. The reporting may also be directly or indirectly to the patient. The reporting may be by any means of communication, including those described above.

The materials and methods of the present invention are ideally suited for preparation of kits produced in accordance with well known procedures. The invention thus provides kits comprising agents (like the polynucleotides and/or antibodies described herein as non-limiting examples) for the detection of expression of HoxB13 sequences. Such kits, optionally comprising the agent with an identifying description or label or instructions relating to their use in the methods of the present invention, are provided. Such a kit may comprise containers, each with one or more of the various reagents (typically in concentrated form) utilized in the methods, including, for example, pre-fabricated microarrays, buffers, the appropriate nucleotide triphosphates (e.g., dATP, dCTP, dGTP and dTTP; or rATP, rCTP, rGTP and UTP), reverse transcriptase, DNA polymerase, RNA polymerase, and one or more primer complexes of the present invention (e.g., appropriate length poly(T) or random primers linked to a promoter reactive with the RNA polymerase). A set of instructions will also typically be included.

Having now generally described the invention, the same will be more readily understood through reference to the following examples which are provided by way of illustration, and are not intended to be limiting of the present invention, unless specified.

### EXAMPLES

### Example 1

### Cell Culture and Cell Line Construction

MCF-10A cells (ATCC; see Soule et al. “Isolation and characterization of a spontaneously immortalized human breast epithelial cell line, MCF-10.” Cancer Res 50:6075-86 (1990)) were maintained in growth medium as described (see Debnath et al. “Morphogenesis and oncogenesis of MCF-10A mammary epithelial acini grown in three-dimensional basement membrane cultures.” Methods 30:256-68 (2003)) in DMEM/F12 (Invitrogen) with 5% horse serum (Invitrogen), 20 ng/ml EGF (Peprotech), 10 μg/ml insulin (Sigma), 100 ng/ml cholera toxin, 0.5 μg/ml hydrocortisone, 50 U/ml penicillin, and 50 μg/ml streptomycin. Assay medium (AM) is identical to the growth medium except 2% instead of 5% horse serum was used.

Human cDNA for HOXB13 in the pDNR plasmid was generously provided by Joshua LaBaer (Harvard Medical School). HOXB13 was subcloned into the SnaB1 site of the retroviral expression vector pBabe-puro (see Morgenstern et al. “Advanced mammalian gene transfer: high titre retroviral vectors with multiple drug selection markers and a complementary helper-free packaging cell line.” Nucleic Acids Res 18:3587-96 (1990)) and proper orientation determined by restriction mapping. Replication incompetent virus with the Vesicular Stomatitis Virus (VSV) envelope was generated from VSV-GPG packaging cells as described (see Ory et al. “A stable human-derived packaging cell line for production of high titer retrovirus/vesicular stomatitis virus G pseudotypes.” Proc Natl Acad Sci., USA 93:11400-6 (1996)), and stable pools of MCF-10A cells were generated by retroviral infection as described (see Debnath et al.) using 2 μg/ml puromycin for selection.

### Example 2

### Transwell Migration and Invasion Assay

In vitro migration and invasion assays were performed using 24 well modified Boyden chamber transwell with PET (polyethylene terephthalate) membranes containing 8 micron pores (BD BioCoat). Uncoated membranes were used for migration assays, and Matrigel (a complex mixture of extracellular matrix components derived from the Engelbroth-Holm-Swarm (EHS) sarcoma) coated membranes were used for invasion (see Repesh et al. “A new in vitro assay for quantitating tumor cell invasion.” Invasion Metastasis 9:192-208 (1989)).

Stable pools of MCF-10A cells infected with retroviral constructs were maintained in growth medium until the day of the assay. 5×10⁴ cells in 100 μl of assay medium were seeded in the upper chamber and 500 μL assay medium with or without 20 ng/ml EGF was added to the lower chamber. Cells were incubated at 37° C. for 24 hours, then fixed in 70% ethanol for 20 minutes, rinsed with PBS, and stained with DAPI (500 ng/ml). Cells that remained on the upper surface were mechanically removed with a cotton swab. Cells remaining on the underside were counted (5 fields at 20× magnification per trans-well). Trans-wells were plated in triplicates and the results were averaged.

Invasion through a Matrigel coated modified Boyden chamber was assayed as described (see Albini et al. “A rapid in vitro assay for quantitating the invasive potential of tumor cells.” Cancer Res 47:3239-45 (1987) and Repesh et al.).

### Example 3

### Results

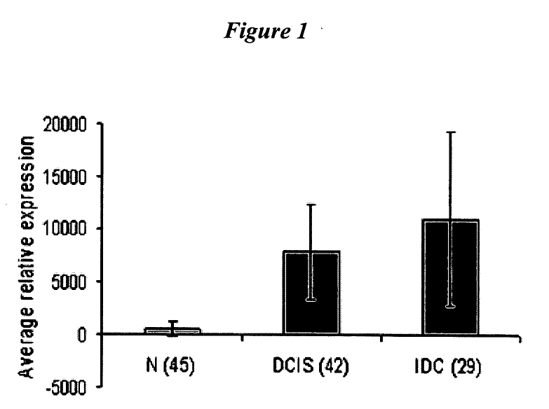

HoxB13 stimulates mammary epithelial cell migration and invasion. As shown in FIG. 1, RT-QPCR analysis of LCM-procured normal and malignant breast epithelial cells from a previously published cohort (n=45, see Ma et al. “Gene expression profiles of human breast cancer progression.” Proc Natl Acad Sci., USA 100:5974-9 (2003)) demonstrated that compared to normal breast epithelial cells, the mean expression levels of HOXB13 were significantly higher in both ductal carcinoma in situ (DCIS, P=0.002) and invasive ductal carcinoma (IDC, P=0.006). Compared to patient-matched normals, 56% DCIS or IDC cases overexpressed HOXB13 by >2-fold.

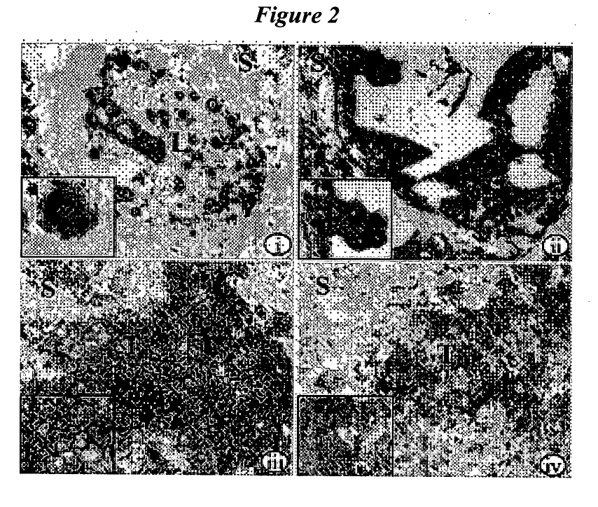

FIG. 2 shows RNA in situ hybridization confirmation of the tumor cell-specific expression of HOXB13 . Interestingly, a subset of normal breast specimens demonstrated expression of HOXB13 in terminal duct lobular unit, raising the possibility that it may play a role in normal mammary physiology.

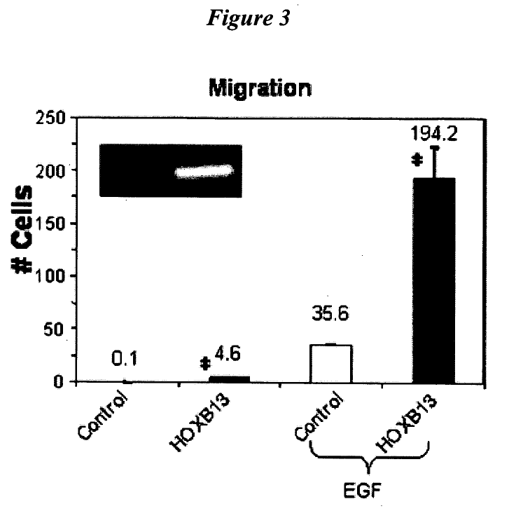

The potential biological function of HOXB13 was studied by expressing a CMV-driven construct in MCF-10A cells. Ectopic expression of HOXB13 in MCF10 was confirmed by RT-QPCR (FIG. 3, insert). Cells expressing HOXB13 displayed distinct morphological changes, characterized by a reduction in epithelial-type junctions (data not shown). Compared to control-infected cells, MCF10A cells expressing HOXB13 had a 5-fold increase in cell motility in trans-well migration assays in the presence of EGF (FIG. 3). Cells expressing HOXB13 also displayed an increase in migration in the absence of exogenously supplied EGF.

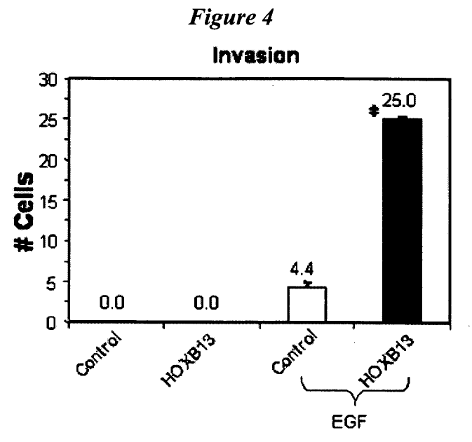

Invasion through a Matrigel coated modified Boyden chamber, a well-established assay correlated with metastatic potential in vivo, was also enhanced 5-fold by HOXB13 expression in the presence of EGF (FIG. 4). Without being bound by theory, these observations suggest that HOXB13 may regulate a pathway that functions synergistically with EGF-dependent signaling to stimulate cell motility and invasion in vitro.

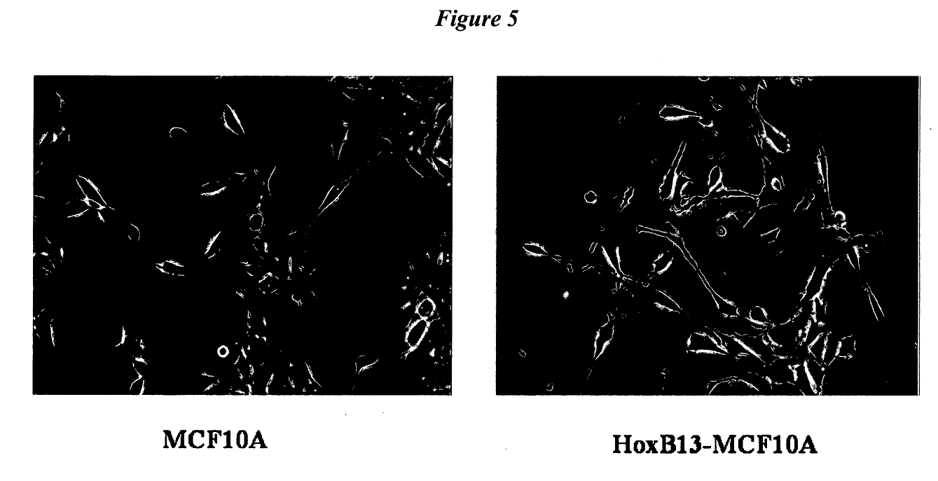

FIG. 5 shows the 2D morphology of MCF10A cells with MCF10A cells that express HOXB13 ectopically.

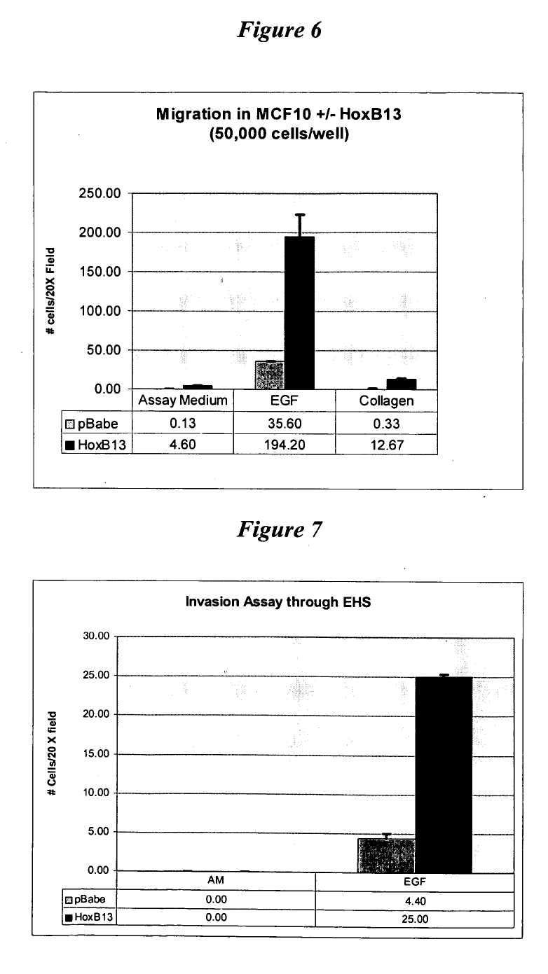

FIG. 6 shows that ectopic HoxB13 expression in MCF10 cells enhances EGF-stimulated migration through extracellular matrix components from EHS (Engelbroth-Holm-Swarm) sarcoma. The inset is as described for FIG. 3. Migration of cells expressing HOXB13 is increased further in the presence of either collagen or EGF.

FIG. 7 shows ectopic HoxB13 expression in MCF10A cells enhances EGF-stimulated invasion through an EHS substrate.

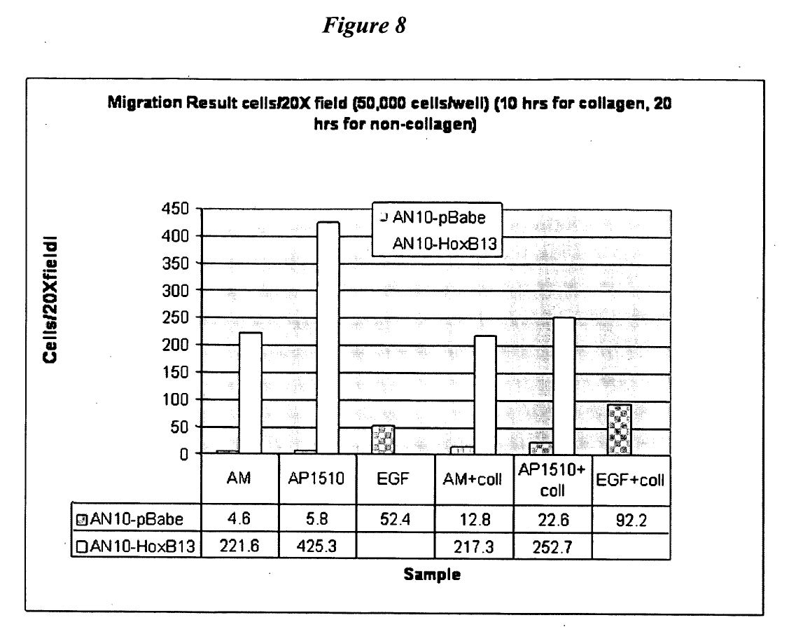

FIG. 8 shows HoxB13 expression in AN10 cells enhances migration with and without a synthetic dimerizer (AP1510). AM denotes assay medium; coll refers to collagen.

All references cited herein, including patents, patent applications, and publications, are hereby incorporated by reference in their entireties, whether previously specifically incorporated or not. However, citation of documents herein is not intended as an admission that any is pertinent prior art. All statements as to the date or representation as to the contents of documents is based on the information available to the applicant and does not constitute any admission as to the correctness of the dates or contents of the documents.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A method of identifying or classifying the potential of one or more sample cells to metastasize into other tissues, said method comprising
determining the level of expression from the HoxB13 gene in said one or more cells, wherein a relatively increased level of expression indicates an increased potential for metastasis in said one or more cells.

2. A method of identifying or classifying the potential of one or more sample cells to invade other tissues, said method comprising
determining the level of expression from the HoxB13 gene in said one or more cells, wherein a relatively increased level of expression indicates an increased potential for invasiveness in said one or more cells.

3. The method of claim 1 wherein said one or more sample cells is of a primary cancer, optionally estrogen receptor positive cancer, of a subject or patient.

4. The method of claim 3 wherein said cancer is selected from Adenocarcinoma of Breast, Adenocarcinoma of Cervix, Adenocarcinoma of Esophagus, Adenocarcinoma of Gall Bladder, Adenocarcinoma of Lung, Adenocarcinoma of Pancreas, Adenocarcinoma of Small-Large Bowel, Adenocarcinoma of Stomach, Astrocytoma, Basal Cell Carcinoma of Skin, Cholangiocarcinoma of Liver, Clear Cell Adenocarcinoma of Ovary, Diffuse Large B-Cell Lymphoma, Embryonal Carcinoma of Testes, Endometrioid Carcinoma of Uterus, Ewings Sarcoma, Follicular Carcinoma of Thyroid, Gastrointestinal Stromal Tumor, Germ Cell Tumor of Ovary, Germ Cell Tumor of Testes, Glioblastoma Multiforme, Hepatocellular Carcinoma of Liver, Hodgkin's Lymphoma, Large Cell Carcinoma of Lung, Leiomyosarcoma, Liposarcoma, Lobular Carcinoma of Breast, Malignant Fibrous Histiocytoma, Medulary Carcinoma of Thyroid, Melanoma, Meningioma, Mesothelioma of Lung, Mucinous Adenocarcinoma of Ovary, Myofibrosarcoma, Neuroendocrine Tumor of Bowel, Oligodendroglioma, Osteosarcoma, Papillary Carcinoma of Thyroid, Pheochromocytoma, Renal Cell Carcinoma of Kidney, Rhabdomyosarcoma, Seminoma of Testes, Serous Adenocarcinoma of Ovary, Small Cell Carcinoma of Lung, Squamous Cell Carcinoma of Cervix, Squamous Cell Carcinoma of Esophagus, Squamous Cell Carcinoma of Larynx, Squamous Cell Carcinoma of Lung, Squamous Cell Carcinoma of Skin, Synovial Sarcoma, T-Cell Lymphoma, and Transitional Cell Carcinoma of Bladder.

5. The method of claim 4 wherein said cancer is of a tissue selected from Adrenal, Bladder, Bone, Brain, Breast, Cervix, Endometrium, Esophagus, Gall Bladder, Kidney, Larynx, Liver, Lung, Lymph Node, Ovary, Pancreas, Prostate, Skin, Soft Tissue, Small/Large Bowel, Stomach, Testes, Thyroid, and Uterus.

6. The method of claim 1, further comprising determining the nodal status of the subject, wherein the absence of cancer in the lymph nodes in combination with an above normal level of HoxB13 expression is used to indicate said increased potential for metastasis.

7. The method of claim 1 wherein said one or more cells are in a biological sample of cells obtained from a subject.

8. The method of claim 7 wherein said sample is a fresh sample, a frozen sample, or a fixed sample.

9. The method of claim 1 wherein said determining comprises assaying the level of HoxB13 mRNA expression, demethylation of HoxB13 DNA, or the level of HOXB13 protein expression.

10. The method of claim 9 wherein said determining comprises assaying mRNA expression by use of quantitative PCR, including reverse transcriptase-PCR and real time PCR.

11. The method of claim 9 wherein said determining comprises assaying mRNA expression by use of a microarray.

12. The method of claim 1 wherein said determining comprises assaying protein expression by detection of a fragment or epitope of an expressed HoxB13 sequence.

13. The method of claim 1 wherein a normal or below normal level of expression indicates the absence of an increased potential, or a decreased potential, for metastasis.

14. A method of predicting the prognosis or disease outcome of a subject, said method comprising
determining the level of expression from the HoxB13 gene in one or more cells of a biological sample obtained from said subject,
wherein an above normal level of expression indicates an increased potential for cancer metastasis, increased likelihood of cancer recurrence, or decreased life expectancy in said subject and a normal or below normal level of HoxB13 expression indicates the absence of an increased potential for cancer metastasis, increased likelihood of cancer recurrence, or decreased life expectancy.

15. The method of claim 14 wherein said cancer recurrence is selected from local recurrence, regional recurrence, distant recurrence, or contralateral recurrence.

16. The method of claim 14 wherein said sample is a pre-cancerous sample or biopsy, or a diagnosed cancer sample or biopsy.

17. A determining the treatment of a subject, said method comprising
determining the prognosis or outcome of said subject by the method of claim 14; and
determining the treatment for said subject based on said prognosis or outcome.

18. The method of claim 14 wherein said cancer recurrence is selected from local recurrence, regional recurrence, distant recurrence, or contralateral recurrence.

19. The method of claim 14 wherein said one or more cells are of a cancer, optionally primary or estrogen receptor positive cancer.

20. The method of claim 2 wherein said cells are breast cells, optionally ADH or DCIS cells.
